(19) United States
(12) Patent Application Publication
(10) Pub. No.: US 2007/0156250 A1
(43) Pub. Date: Jul. 5, 2007

(54) Orthopedic Implants Coated with Pyrolytic Carbon

(76) Inventors: William H. Seitz Jr., Shaker Heights, OH (US); Albert N. Santilli, Pepper Pike, OH (US)

Correspondence Address: RANKIN, HILL, PORTER & CLARK, LLP, 925 EUCLID AVENUE, SUITE 700, CLEVELAND, OH, 44115-1405, US

(21) Appl. No.: 11/608,454

(22) Filed: Dec. 8, 2006

Related U.S. Application Data
(60) Provisional application No. 60/749,131, filed on Dec. 9, 2005.

(51) Int. Cl. A61F 2/28; A61F 2/36; A61F 2/40

(52) U.S. Cl. 623/23.51; 623/23.14; 623/23.13; 427/2.26; 623/19.14

(57) ABSTRACT

An orthopedic implant having a metal substrate with an articulating surface includes a coating for the articulating surface made of pyrolytic carbon. Typically, the substrate is made of a metal such as titanium or stainless steel, or alloys thereof. The pyrolytic carbon coating preferably is applied by vapor deposition.

### BACKGROUND OF THE INVENTION

1. Field of the Invention

The present invention relates generally to orthopedic implants and, more particularly, to orthopedic implants having a coating of pyrolytic carbon thereon.

2. Description of the Prior Art

Orthopedic implants currently are made of many different types of materials. Some implants are made of ultra-high molecular weight polyethylene, while others are made of biocompatible materials such as titanium, titanium alloys, surgical alloys, stainless steels, ceramics, and cobalt chrome. It is desirable for an orthopedic implant to be made of material that has properties similar to bone, that is resistant to wear from frictional forces, and that is durable and strong. It also is important for orthopedic implant materials to be biocompatible so as to minimize any adverse effects on the patient's body when the implant is placed therein. Accordingly, some implants have been coated with special material that is thought to be relatively biocompatible. For example, some implants have a porous coating made from powdered materials such as a titanium alloy. Some implants are coated with hydroxyapatite, a calcium phosphate type of ceramic.

In the particular case of shoulder implants, the end or head of the humerus bone either is replaced or is covered (resurfaced) by a mushroom-shaped implant inserted therein. The implant often is designed to cooperate with a concave-shaped piece placed at the glenoid cavity of the scapula to receive the implant coupled to the humerus. Typical shoulder implants are disclosed in U.S. Pat. Nos. 4,865,605; 5,800,551; 5,807,407; 6,364,910; and 6,783,549, the disclosures of which are incorporated herein by reference.

Similarly, in elbow implants, the end or head of the radius bone is removed and replaced by an implant having a generally concave head at the end thereof. See, for example, U.S. Pat. Nos. 6,217,616 and 6,656,225, the disclosures of which are incorporated herein by reference.

Existing implants are susceptible to wear and tear due to frictional forces, particularly those that work on the exterior of the implant coupled to mating portions of the patient's body or other implants (articulating surface). Existing implants also are susceptible to blood clots that form on the surface of the implants. These blood clots can pose extreme danger to the patient. In view of the noted drawbacks of known orthopedic implants, it would be highly desirable to have a low-friction implant that would minimize or avoid the tendency to form blood clots, as well as being able to resist wear and tear. Any such implant desirably has a head or other articulating surface that is made of a strong biocompatible material such as metal or a metal alloy made of titanium, stainless, steel, or the like.

### SUMMARY OF THE INVENTION

In response to the foregoing concerns, the present invention provides a new and improved orthopedic implant. The orthopedic implant according to the present invention comprises a metal substrate coated on an articulating surface with pyrolytic carbon or an alloy of pyrolytic carbon. The invention can be used with virtually any type of orthopedic implant. In one illustrative form of the present invention, a resurfacing shoulder implant comprises a head that is designed to fit over at least a portion of the proximal end of the humerus bone. The implant according to the invention further comprises a stem, preferably fenestrated, for insertion into an opening in the humerus bone. The head is comprised of a cap having a porous coating. Typically, the cap is made of a metal such as titanium or stainless steel, or alloys thereof. The coating over the cap comprises pyrolytic carbon or an alloy thereof, preferably applied by vapor deposition.

The orthopedic implants of the present invention enjoy significant advantages that existing orthopedic implants do not. The pyrolytic coating on the articulating surface makes the implants of the present invention resistant to friction and wear, resistant to blood clotting, resistant to adverse reactions when implanted into a human body, and resistant to damage of surrounding cartilage. The use of a metallic substrate provides a strong, biocompatible material for the body of the implant. The substrate also has excellent adhesion characteristics for pyrolytic carbon. The foregoing features and advantages will be apparent from the accompanying drawings and the description that follow.

### BRIEF DESCRIPTION OF THE DRAWINGS

FIG. 1 illustrates a resurfacing shoulder implant, in accordance with one embodiment of the present invention, that has been implanted at the head of a humerus bone;

FIG. 2 is a perspective view of an implant according to one embodiment of the present invention;

FIG. 3 is an exploded perspective view of the implant shown in FIG. 2;

FIG. 4 is an exploded elevational view of the implant shown in FIG. 2; and

FIG. 5 depicts sample sizes for a head of the implant in accordance with some embodiments of the present invention.

### DESCRIPTION OF THE PREFERRED EMBODIMENTS

Referring to FIG. 1, an implant 10 according to the present invention is shown. The implant 10 is intended to be implanted into the head of a humerus bone 12. Unlike a complete shoulder implant, the implant 10 is a resurfacing implant that is designed to cover or cap only the top portion of the humerus bone 12.

Although the invention is described herein in the context of a resurfacing shoulder implant 10, it is to be understood that the description of the invention in such an application is for illustrative purposes only. The present invention is applicable to virtually all types of orthopedic implants, including radial head implants, basal thumb implants, spinal implants, etc.

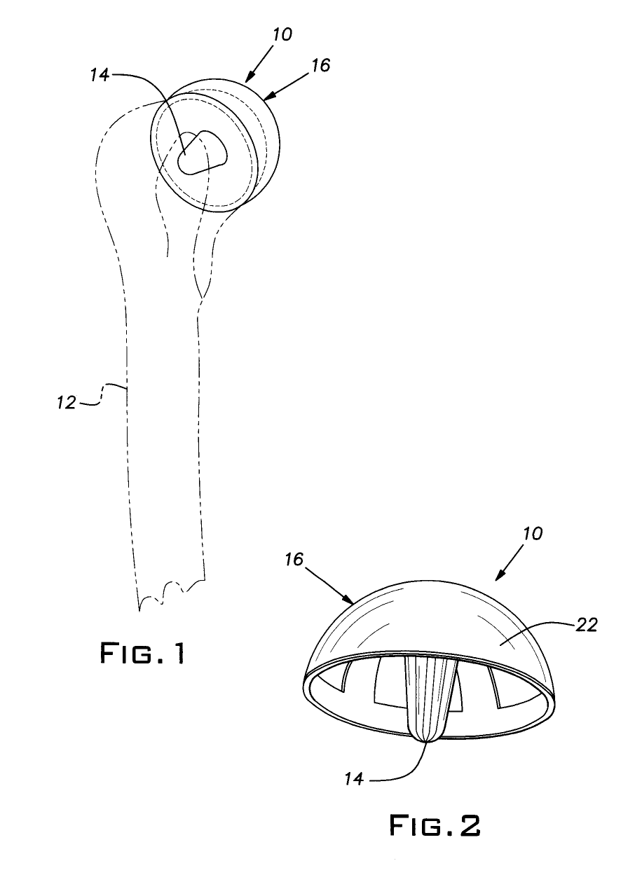

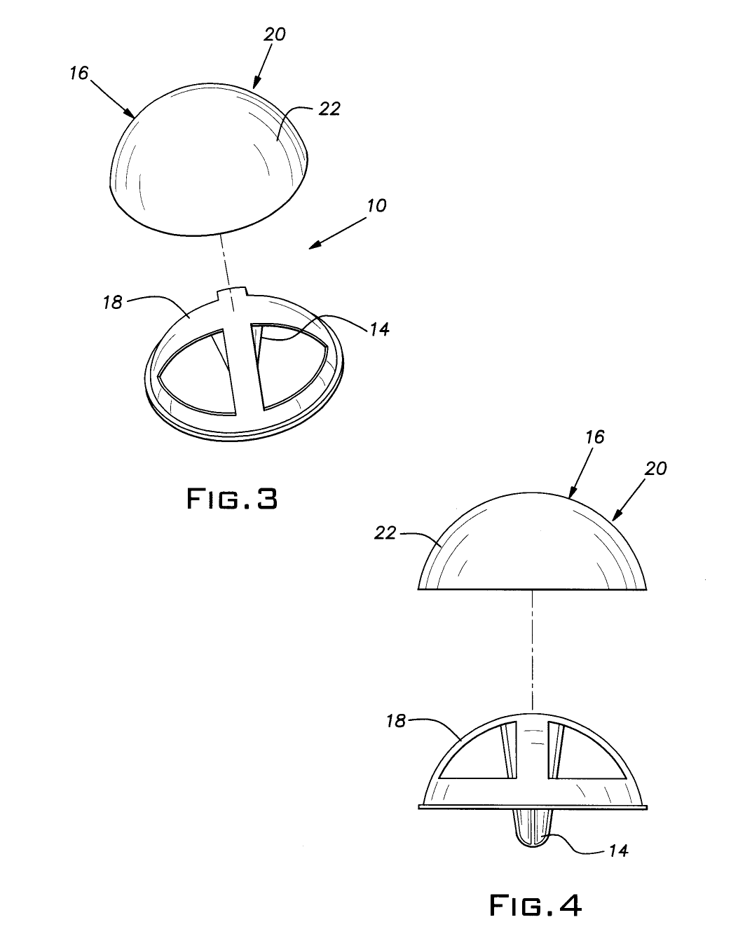

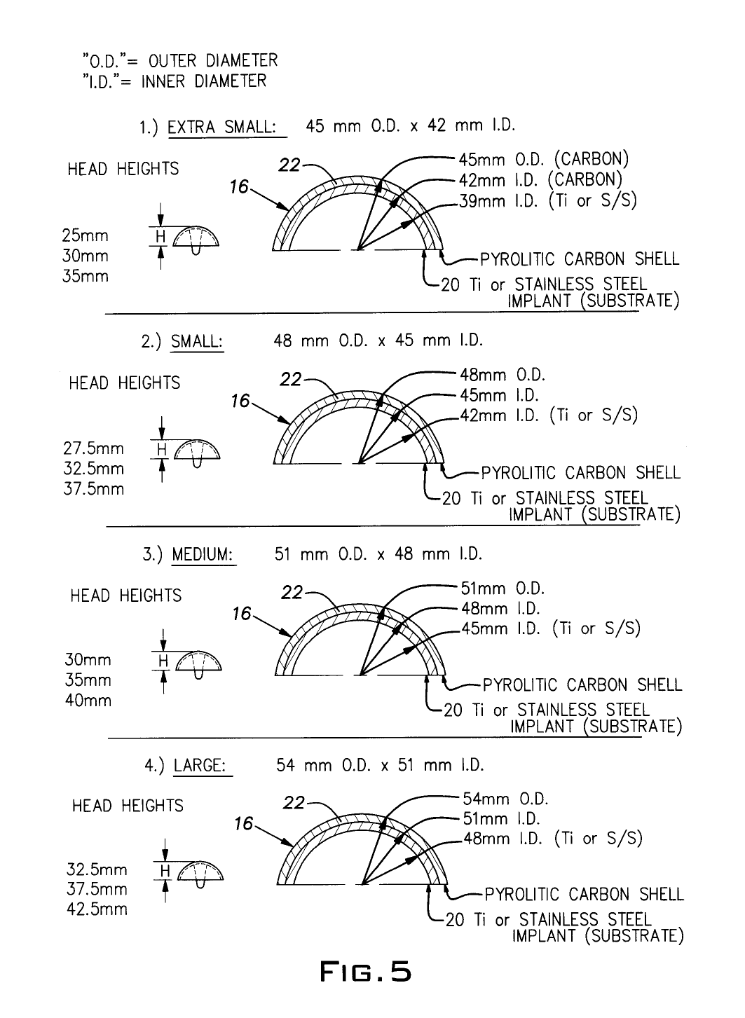

The implant 10 comprises a dome or head 16 and a stem 14. FIGS. 2 through 4 illustrate, via multiple perspectives, various parts of the implant 10. FIG. 2 is a perspective view of one embodiment of the implant 10, FIG. 3 is an exploded perspective view of the implant 10 shown in FIG. 2, and FIG. 4 is an exploded elevational view of the implant 10 shown in FIG. 2.

Head 16 is a dome-like member that includes a cap or substrate 20 (see FIG. 5). The head 16 is intended to fit over at least a portion of the end of the humerus bone. The cap 20 may be any type of suitable material for a resurfacing shoulder implant, but the preferred materials are titanium, stainless steel, or alloys thereof. The cap 20 is further coated with a layer 22 of pyrolytic carbon, as will be further explained herein. In the preferred embodiments of the present invention, the head 16 is manufactured in multiple sizes, each size having specific diameters and heights. FIG. 5, for example, shows four preferred sizes, including the corresponding dimensions for the thickness of the layers 22 of pyrolytic carbon. It shall be noted, however, that implants 10 of other sizes and shapes are within the scope of the present invention. For example, rather than the thickness of the caps 20 being 3 mm as illustrated, the thickness may be 2 mm, etc.

Stem 14 is a projection or extension designed to be inserted into an opening made in the head of the patient's humerus bone 12. In preferred embodiments of the present invention, the stem 14 is fenestrated to promote bone growth therein. In some embodiments of the present invention, the cap 20 and the stem 14 are initially separate pieces; in other embodiments, they are formed integrally. Moreover, as can be seen best in FIGS. 3 and 4, stem 14 may be coupled to a seat 18 upon which the cap 16 is seated. FIG. 2 illustrates the configuration of the cap 16 and the seat 18 when the cap 16 is seated upon the seat 18. It will be noted that the drawings depict the seat 18 as generally umbrella-shaped. However, seats 18 of other different shapes are within the scope of the present invention.

The pyrolytic carbon layer 22 used in the present invention can be either an alloy or a pure pyrolytic carbon. All such materials are encompassed by the term “pyrolytic carbon” as used herein. Pyrolytic carbon has many properties that make it particularly advantageous for use as a coating on a resurfacing shoulder implant. For example, pyrolytic carbon is resistant to friction and wear, thromboresistant (that is, it resists the tendency to cause blood to clot at its surface), and biocompatible (that is, it does not cause adverse reactions when implanted into a human body). Moreover, unlike implants having surfaces of metal or ceramic, implants 10 coated with a layer 22 of pyrolytic carbon do not tend to damage surrounding cartilage.

The pyrolytic carbon layer 22 is formed by chemical vapor deposition. In particular, hydrocarbon is heated to a gaseous state at temperatures typically ranging from about 1000 to 2500 K. The hydrocarbon gas then is allowed to crystalize onto an underlying cap or substrate 20. One particular type of pyrolytic carbon that may be used in the present invention is the On-X brand of pyrolytic carbon marketed by Medical Carbon Research Institute of Austin, Tex. For a more complete description of processes and apparatus for the deposition of pyrolytic carbon on substrates, reference is made to U.S. Pat. Nos. 5,284,676 and 6,410,087, the disclosures of which are incorporated herein by reference.

Although the invention has been described in its preferred form with a certain degree of particularity, it will be understood that the present disclosure of the preferred embodiments has been made only by way of example and that various changes may be resorted to without departing from the true spirit and scope of the invention as hereinafter claimed.

What is claimed is:

1. An orthopedic implant, comprising:
a metal substrate that defines an articulating surface; and
a coating of pyrolytic carbon on the articulating surface.

2. The implant of claim 1, wherein the substrate is contoured to fit a mating portion of a patient's body.

3. The implant of claim 1, wherein the metal substrate is selected from the group consisting of titanium, stainless steel, cobalt chrome steel, and alloys and mixtures thereof.

4. The implant of claim 1, wherein the pyrolytic carbon is On-X brand pyrolytic carbon.

5. The implant of claim 1, wherein the pyrolytic carbon coating has a thickness within the range of 2-3 mm.

6. An orthopedic implant, comprising:
a metal substrate that defines an articulating surface that is contoured to fit a mating portion of a patient's body, the metal substrate being selected from the group consisting of titanium, stainless steel, cobalt chrome steel, and alloys and mixtures thereof; and
a coating of pyrolytic carbon on the articulating surface, the pyrolytic carbon coating having a thickness within the range of 2-3 mm.

7. The implant of claim 6, wherein the pyrolytic carbon is On-X brand pyrolytic carbon.

8. A method of manufacturing an orthopedic implant having an articulating surface, comprising the steps of:
providing a metal substrate that defines the articulating surface; and
applying a coating of pyrolytic carbon on the articulating surface.

9. The method of claim 8, wherein the metal substrate is selected from the group consisting of titanium, stainless steel, cobalt chrome steel, and alloys and mixtures thereof.

10. The method of claim 8, wherein the pyrolytic carbon is On-X brand pyrolytic carbon.

11. The method of claim 8, wherein the pyrolytic carbon is applied to a thickness within the range of 2-3 mm.
